SEQUENCE LISTING (8 sequences)

SEQ ID NO: 1 - DNA, 19 bp, synthetic construct
  source 1..19: mol_type = other DNA
ggagatcgagctcgcctat

SEQ ID NO: 2 - DNA, 19 bp, synthetic construct
  source 1..19: mol_type = other DNA
catcggtacttgagcaatg

SEQ ID NO: 3 - RNA, 19 nt, synthetic construct
  source 1..19: mol_type = other RNA
ggcaaaggccctcaaataa

SEQ ID NO: 4 - RNA, 19 nt, synthetic construct
  source 1..19: mol_type = other RNA
ggaccgagacagtgcagaa

SEQ ID NO: 5 - RNA, 19 nt, synthetic construct
  source 1..19: mol_type = other RNA
ccaaaagactcaccaagta

SEQ ID NO: 6 - RNA, 19 nt, synthetic construct
  source 1..19: mol_type = other RNA
cctcatagcttaaagtata

SEQ ID NO: 7 - RNA, 19 nt, synthetic construct
  source 1..19: mol_type = other RNA
tgacttagctatagcagta

SEQ ID NO: 8 - RNA, 19 nt, synthetic construct
  source 1..19: mol_type = other RNA
gacaaaagctagctagtaa

(19) United States
(12) Patent Application Publication
(10) Pub. No.: US 2023/0381503 A1
(43) Pub. Date: Nov. 30, 2023

(54) Compositions And Methods For Treating With A Combination Of Alternating Electric Fields And Ion Channel Inhibitors

(71) Applicant: NOVOCURE GMBH, Root D4 (CH)

(72) Inventors: Stuart Smith, Nottingham (GB); Michele Mazzanti, Milano (IT)

(21) Appl. No.: 18/193,837

(22) Filed: Mar. 31, 2023

Related U.S. Application Data
(60) Provisional application No. 63/351,327, filed on Jun. 10, 2022.
(60) Provisional application No. 63/326,075, filed on Mar. 31, 2022.

(51) Int. Cl. A61N 1/32; A61K 31/192; A61P 35/00

(52) U.S. Cl. CPC A61N 1/32; A61K 31/192; A61P 35/00

(57) ABSTRACT

Disclosed are methods of treating a subject having cancer comprising applying alternating electric fields to a target site of the subject for a period of time, the alternating electric fields having a frequency and field strength, wherein the target site comprises one or more cancer cells, and administering an ion channel inhibitor to the subject. Disclosed are methods of killing a cancer cell comprising exposing the cancer cell to alternating electric fields for a period of time, the alternating electric fields having a frequency and field strength, and contacting the cancer cell with an ion channel inhibitor, wherein cancer cells are killed. Disclosed are methods of reducing cancer cell proliferation comprising exposing the cancer cell to alternating electric fields for a period of time, the alternating electric fields having a frequency and field strength, and contacting the cancer cell with an ion channel inhibitor, wherein the alternating electric fields and ion channel inhibitor provide an anti-proliferative effect of the cancer cell. Disclosed are methods of increasing survival rate of a subject having cancer comprising applying alternating electric fields to a target site of the subject for a period of time, the alternating electric fields having a frequency and field strength, wherein the target site comprises one or more cancer cells, and administering an ion channel inhibitor to the subject, wherein the subject has an increased survival rate.

### CROSS-REFERENCE TO RELATED APPLICATIONS

This application claims the benefit of U.S. Provisional Patent Application No. 63/326,075, filed Mar. 31, 2022 and U.S. Provisional Patent Application No. 63/351,327, filed Jun. 10, 2022, each of which is incorporated by reference herein in their entirety.

### BACKGROUND

Glioblastoma multiforme (GBM) is a lethal brain cancer with an average survival of 14 months, forming the leading cause of CNS cancer-related death in children. There are currently no curative treatments. Tumour treating fields (TTFields) is a non-invasive antimitotic therapy approved in GBM patients>22 years. Previous studies have identified the clinical

benefit of TTFields; however, the mechanistic action is unclear. Thus, TTFields are an effective and safe treatment modality for glioblastoma patients increasingly offered to patients in good performance status willing to wear the electrode arrays.

Increasing evidence suggests that ion channels not only regulate electrical signalling of excitable cells, but they also play a crucial role in the development and progression of brain tumours. Ion channels are essential in cell cycle control, invasion, and migration of cancer cells and therefore present as valuable therapeutic targets.

Both potassium selective pores and chloride permeabilities are considered the most active channels during tumorigenesis. High rate of proliferation, active migration, and invasiveness into non-neoplastic tissues are specific properties of neoplastic transformation. All these actions require partial or total involvement of chloride channel activity.

### BRIEF SUMMARY

Disclosed herein are methods comprising combining TTFields with ion channel inhibitors (e.g., a chloride ion channel inhibitor) to treat cancer.

Disclosed are methods of treating a subject having cancer comprising applying alternating electric fields to a target site of the subject for a period of time, the alternating electric fields having a frequency and field strength, wherein the target site comprises one or more cancer cells, and administering an ion channel inhibitor (e.g., a chloride ion channel inhibitor) to the subject.

Disclosed are methods of killing a cancer cell comprising exposing the cancer cell to alternating electric fields for a period of time, the alternating electric fields having a frequency and field strength, and contacting the cancer cell with an ion channel inhibitor (e.g., a chloride ion channel inhibitor), wherein cancer cells are killed.

Disclosed are methods of reducing cancer cell proliferation comprising exposing the cancer cell to alternating electric fields for a period of time, the alternating electric fields having a frequency and field strength, and contacting the cancer cell with an ion channel inhibitor (e.g., a chloride ion channel inhibitor), wherein the alternating electric fields and ion channel inhibitor provide an anti-proliferative effect of the cancer cell.

Disclosed are methods of increasing survival rate of a subject having cancer comprising applying alternating electric fields to a target site of the subject for a period of time, the alternating electric fields having a frequency and field strength, wherein the target site comprises one or more cancer cells, and administering an ion channel inhibitor (e.g., a chloride ion channel inhibitor) to the subject, wherein the subject has an increased survival rate.

Also disclosed are any of the methods described herein further comprising a step of administering a second therapeutic, such as a chemotherapeutic agent.

Additional advantages of the disclosed methods and compositions will be set forth in part in the description which follows, and in part will be understood from the description, or may be learned by practice of the disclosed methods and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and, together with the description, serve to explain the principles of the disclosed methods and compositions.

FIGS. 1A and 1B show mRNA (A) and protein (B) levels of CLIC1 and CLIC4 are over expressed in pediatric GBM (pGBM) compared to non-neoplastic human astrocytes (HA). A) Standard real time PCR measurement of mRNA levels for the gene (CLIC1 or CLIC4) in the cell lines. Also shown is RNA sequencing of GBM patient tissue. KNS42 and SF188 are widely available pediatric high grade glioma cell lines. GCE62 is an in house low passage GBM line from a teenager. B) Semiquantified protein expression on IHC compared to normal placenta as control.

FIG. 2 shows the overexpression of CLIC1 and CLIC4 confers poor overall survival in patient cohorts.

FIG. 3 shows an example of pharmacological inhibition via treatment with 100 uM IAA94 reduces overall cell count and reduces invasive capacity in pGBM. The cell count in different cell lines (SF188 and KNS42) with and without IAA94.

FIG. 4 shows TTFields have efficacy in significantly reducing pGBM cell proliferation in two cell lines (SF188 and KNS42). The Presto Blue metabolic viability test was used.

FIG. 5 shows treatment of two cell lines (KNS42 and SF188) with TTFields plus the knock down of CLIC1 or CLIC4. CLIC1 and CLIC4 knock down significantly increased sensitivity to TTFields, reducing cell proliferation post treatment at 200 kHz when compared to 200 kHz alone.

FIG. 6 shows treatment of three cell lines (GCE62, SF188 and KNS42) with TTFields and IAA94.

FIGS. 7A and 7B show an increase in CLIC1 and CLIC4 expression. A) Immunoflourescent staining against CLIC1 and CLIC4 reveals an over expression in paediatric glioma cell lines when normalised to human astrocytes. Membranous staining can be observed in SF188 and GCE62 cell. B) Staining via immunohistochemistry reveals a significant cytoplasmic expression pattern when compared to temporal lobe tissue.

FIGS. 8A-8B show treatment with electrotherapy causes CLIC1 and CLIC4 deficiency. A) rtPCR of pGBM cells reveal CLIC1 and CLIC4 expression is significant reduced following treatment with TTFields, whereas P2RX7 and P2RX4 are upregulated in SF188. B) Transcriptome analysis of pGBM reveals a unique ion channel signatures post TTFields treatment at 200 kHz. CLIC1 and CLIC4 are significantly down regulated in treatment groups vs controls.

FIG. 9 shows CLIC1 and CLIC4 knock down+200 kHz TTFields significantly reduces clonogenic capacity in SF188 when compared to 200 kHz alone.

FIGS. 10A-10B show CL1C1 and CLIC4 deficiency reduces the capacity of pGBM to proliferate, invade and migrate. A) Colony formation is significantly reduced when CLIC1 and CLIC4 are knocked down. B) CLCI1 and CLIC4 knock down via siRNA significantly reduced the ability of pGBM to invade.

FIG. 11 shows mRNA expression of CLIC1 at 72 and 288 hours of TTFields exposure.

FIG. 12 shows the combination of TTFields plus the CLIC1 inhibitor, IAA94, on cell number.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a growth factor inhibitor” includes a plurality of such growth factor inhibitors, reference to “the cell” is a reference to one or more cells and equivalents thereof known to those skilled in the art, and so forth.

As used herein, a “target site” is a specific site or location within or present on a subject or patient. For example, a “target site” can refer to, but is not limited to a cell (e.g. a cancer cell), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, the brain. In some aspects, a cell or population of cells that can be a target site or a target cell include, but are not limited to, a cancer cell (e.g. a glioblastoma cell). In some aspects, a “target site” can be a tumor target site.

A “tumor target site” is a site or location within or present on a subject or patient that comprises or is adjacent to one or more cancer cells, previously comprised one or more tumor cells, or is suspected of comprising one or more tumor cells. For example, a tumor target site can refer to a site or location within or present on a subject or patient that is prone to metastases. Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g. a target site such as a cell). In some aspects, the alternating electrical field can be in a single direction or multiple directional. In some aspects, alternating electric fields can be delivered through two pairs of transducer arrays that generate perpendicular fields within the target site. For example, for the Optune™ system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

As used herein, an “alternating electric field” applied to a tumor target site can be referred to as a “tumor treating field” or “TTField.” TTFields have been established as an antimitotic cancer treatment modality because they interfere with proper micro-tubule assembly during metaphase and eventually destroy the cells during telophase, cytokinesis, or subsequent interphase. TTFields target solid tumors and is described in U.S. Pat. No. 7,565,205, which is incorporated herein by reference in its entirety for its teaching of TTFields.

In-vivo and in-vitro studies show that the efficacy of TTFields therapy increases as the intensity of the electrical field increases. Therefore, optimizing array placement on a subject to increase the intensity in the target site or target cell is standard practice for the Optune system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g. an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field. For example, the subject of administration can be a subject with glioblastoma.

By “treat” is meant to administer or apply a therapeutic, such as alternating electric fields and a vector, to a subject, such as a human or other mammal (for example, an animal model), that has glioblastoma or has an increased susceptibility for developing glioblastoma, in order to prevent or delay a worsening of the effects of the disease or infection, or to partially or fully reverse the effects of glioblastoma. For example, treating a subject having glioblastoma can comprise delivering a therapeutic to a cell in the subject.

By “prevent” is meant to minimize or decrease the chance that a subject develops glioblastoma.

As used herein, the terms “administering” and “administration” refer to any method of providing an ion channel inhibitor to a subject or directly or indirectly to a target site. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat brain cancer. In further various aspects, a preparation can be administered prophylactically; that is, administered for prevention of brain cancer. In an aspect, the skilled person can determine an efficacious dose, an efficacious schedule, or an efficacious route of administration so as to treat a subject. In some aspects, administering comprises exposing or applying. Thus, in some aspects, exposing a target site or subject to alternating electrical fields or applying alternating electrical fields to a target site or subject means administering alternating electrical fields to the target site or subject.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise alternating electric fields. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 50 and 1 MHz. In some aspects, the frequency of the alternating electric field is between 100 and 500 kHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric fields can be electric fields at 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 kHz.

In some aspects, the field strength of the alternating electric fields can be between 0.5 and 4 V/cm RMS. In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some embodiments the field strength is at least 1 V/cm RMS. In some aspects, the field strength can be 0.9 V/cm RMS. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more.

The disclosed methods comprising applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

### C. Compositions

Disclosed are compositions and formulations comprising one or more ion channel inhibitors, or a combination thereof, with a pharmaceutically acceptable carrier or diluent. In some aspects, the ion channel inhibitor is a chloride ion channel inhibitor. For example, disclosed are pharmaceutical compositions, comprising a chloride ion channel inhibitor and a pharmaceutically acceptable carrier. Chloride ion channel inhibitors can be one or more of those described in Griffin et al. Ion Channels as Therapeutics Targets in High Grade Gliomas. Cancers 2020, 12, 3068, incorporated by reference in its entirety herein.

Disclosed are compositions and formulations comprising a chloride ion channel inhibitor with a pharmaceutically acceptable carrier or diluent. For example, disclosed are pharmaceutical compositions, comprising IAA94, and a pharmaceutically acceptable carrier or diluent.

In some aspects, the chloride ion channel inhibitor can be administered with a pharmaceutically acceptable carrier or diluent in any of the disclosed methods.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl choline (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, P A 1995, which is hereby incorporated by reference for its teaching of pharmaceutically acceptable carriers. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anaesthetics, and the like. In the methods described herein, delivery of the disclosed compositions to cells can be via a variety of mechanisms. The pharmaceutical composition may be administered in a number of ways depending on whether local or systemic treatment is desired, and on the area to be treated.

1. Delivery of Compositions

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

### D. Methods of Treating

Disclosed herein is the use of alternating electric fields to treat glioblastoma. Disclosed herein is the inhibition of chloride ion channels to treat glioblastoma by preventing or decreasing ion flux resulting in cell apoptosis. Alternating electric fields can also downregulate chloride ion channels. Disclosed herein is the use of chloride ion channel inhibitors to enhance the effects of alternating electric fields in the treatment of cancers, such as glioblastoma.

The disclosed methods comprise administering an ion channel inhibitor or a composition comprising an ion channel inhibitor as described throughout. In some aspects, the ion channel inhibitor is a chloride ion channel inhibitor.

Disclosed are methods of treating a subject having cancer comprising applying alternating electric fields to a target site of the subject for a period of time, the alternating electric fields having a frequency and field strength, wherein the target site comprises one or more cancer cells, and administering an ion channel inhibitor to the subject. In some aspects, the ion channel inhibitor is a chloride ion channel inhibitor. Thus, disclosed are methods of treating a subject having cancer comprising applying alternating electric fields to a target site of the subject for a period of time, the alternating electric fields having a frequency and field strength, wherein the target site comprises one or more cancer cells, and administering a chloride ion channel inhibitor to the subject.

In some aspects, the ion channel inhibitor is a chloride ion channel inhibitor. In some aspects, the chloride ion channel inhibitor is an intracellular chloride ion channel (CLIC) inhibitor. In some aspects, the CLIC inhibitor is a CLIC1 or CLIC4 inhibitor. CLIC1 and CLIC 4 have been noted as potential biomarkers in ovarian and colorectal cancer and are over expressed in many tumours, linking to poor overall survival. CLIC1 and CLIC are linked to migratory capacity of glioblastoma cells. Therefore, inhibiting CLIC1 and CLIC4 in combination with treating with alternating electric fields can be an effective therapeutic. In some aspects, a chloride ion channel inhibitor can be, but is not limited to, Niflumic acid, DPC, DIDS, anthracene derivatives, stilbenes, and heavy metal ions blockbenzimidazole, Clofubric acid, Benzofurans, propionic acid (CPP), SITS, NPPB, Chlorotoxin, Mibefradil, Calix[4]arene, Clomiphene, Cd2+, Gd3+, Glibenclaimide, Flufenamic acid, Inositol-tetrabisphosphate, Mefloquinand or Fluoxetine.

In some aspects, the CLIC inhibitor can be a synthetic small molecule, protein, peptide, antibody, aptamer or a nucleic acid. For example, in some aspects, the CLIC inhibitor can be a small molecule, such as IAA94. In some aspects, the CLIC inhibitor is a siRNA. In some aspects, the siRNA inhibits the expression of a protein selected from the group consisting of CLIC1, CLIC4, CLIC2, CLIC3, CLIC5, and CLIC6 For example, in some aspects, CLIC3 is implicated in promoting cell invasion, and increased CLIC3 levels in the stroma can be associated with increased cell invasion capabilities. Thus, inhibiting a CLIC, such as CLIC3, can be a cancer therapeutic. In some aspects, the siRNA can be one or more of GGAGAUCGAGCUCGCCUAU (SEQ ID NO:1), CAUCGGUACUUGAGCAAUG (SEQ ID NO:2), GGCAAAGGCCCUCAAAUAA (SEQ ID NO:3), GGACCGAGACAGUGCAGAA (SEQ ID NO:4), CCAAAAGACUCACCAAGUA (SEQ ID NO:5), CCUCAUAGCUUAAAGUAUA (SEQ ID NO:6), UGACUUAGCUAUAGCAGUA (SEQ ID NO:7), or GACAAAAGCUAGCUAGUAA (SEQ ID NO:8), or the complement thereof. In some aspects, the siRNA can be a sequence comprising 80%, 85%, 90%, 95%, 99% or any percent in-between, identity or complementarity to one or more GGAGAUCGAGCUCGCCUAU (SEQ ID NO:1), CAUCGGUACUUGAGCAAUG (SEQ ID NO:2), GGCAAAGGCCCUCAAAUAA (SEQ ID NO:3), GGACCGAGACAGUGCAGAA (SEQ ID NO:4), CCAAAAGACUCACCAAGUA (SEQ ID NO:5), CCUCAUAGCUUAAAGUAUA (SEQ ID NO:6), UGACUUAGCUAUAGCAGUA (SEQ ID NO:7), or GACAAAAGCUAGCUAGUAA (SEQ ID NO:8).

In some aspects, the ion channel inhibitor can be administered prior to applying the alternating electric fields. In some aspects, administering the ion channel prior to applying the alternating electric fields can refer to administering the ion channel inhibitor at least 1, 2, 3, 4, 5, 6, 7, 8, 9, 10, 11, 12, 13, 14, 15, 16, 17, 18, 19, 20, 21, 22, 23, or 24 hrs prior to or at least 1, 2, 3, 4, 5, 6, 7, 8, 9, or 10 days prior to applying the alternating electric fields. In some aspects, the ion channel inhibitor can be administered after applying the alternating electric fields. In some aspects, administering the ion channel after applying the alternating electric fields can refer to administering the ion channel inhibitor at least 1, 2, 3, 4, 5, 6, 7, 8, 9, 10, 11, 12, 13, 14, 15, 16, 17, 18, 19, 20, 21, 22, 23, or 24 hrs after or at least 1, 2, 3, 4, 5, 6, 7, 8, 9, or 10 days after applying the alternating electric fields. In some aspects, the ion channel inhibitor is administered simultaneously with applying the alternating electric fields. In some aspects, applying the alternating electric fields simultaneously can mean applying within 5, 10, 15, 20, 25, 30, 35, 40, 45, 50, 55, or 60 minutes before or after administering an ion channel inhibitor.

In some aspects, the alternating electric fields and ion channel inhibitor increase overall survival of the subject. In some aspects, the alternating electric fields and ion channel inhibitor reduce proliferation of the one or more cancer cells. In some aspects, the alternating electric fields and ion channel inhibitor reduce migration of the one or more cancer cells. The decrease in proliferation and/or migration of cancer cells can result in a less invasive cancer.

In some aspects, the cancer is glioblastoma. In some aspects, the cancer can be, but is not limited to, brain cancer, breast cancer, ovarian cancer, lung cancer, colon cancer, prostate cancer, sarcoma, colorectal cancer, liver cancer, skin cancer, or stomach cancer.

In some aspects, the frequency of the alternating electric field is between 50 and 1 MHz. In some aspects, the frequency of the alternating electric field is 100-500 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 1 V/cm RMS. In some aspects, the alternating electric field has a field strength of any of those described herein.

### E. Methods of Killing Cells

Alternating electric fields alone and ion channel inhibitors alone can kill cancer cells. The combination of the two can be used to enhance the effects (e.g. increased cell killing) of the alternating electric fields. Alternating electrical fields can increase expression of CLIC1 and CLIC4, which are known to be upregulated in cancer and regulate cell proliferation and apoptosis. In some aspects, as disclosed herein, combining TTFields with CLIC1 or CLIC4 inhibitors can lead to increased cytotoxicity (e.g. killing) in cancer cells.

In some aspects, the disclosed methods comprise contacting a cell with an ion channel inhibitor or a composition comprising an ion channel inhibitor as described throughout.

Disclosed are methods of killing a cancer cell comprising exposing the cancer cell to alternating electric fields for a period of time, the alternating electric fields having a frequency and field strength, and contacting the cancer cell with an ion channel inhibitor, wherein cancer cells are killed. Specifically, disclosed are methods of killing a cancer cell comprising exposing the cancer cell to alternating electric fields for a period of time, the alternating electric fields having a frequency and field strength, and contacting the cancer cell with a chloride ion channel inhibitor, wherein cancer cells are killed.

In some aspects, contacting a cancer cell with a chloride ion channel inhibitor can include adding a chloride ion channel inhibitor to a sample comprising one or more cancer cells. In some aspects, contacting a cancer cell with a chloride ion channel inhibitor can include adding one or more cancer cells to a sample comprising a chloride ion channel inhibitor. Thus, in some aspects, a cancer cell is contacting an ion channel inhibitor and in some aspects, the ion channel inhibitor is contacting a cancer cell. In some aspects, the ion channel inhibitor directly interacts with an ion channel present in or on a cell when the cell is contacted with the ion channel inhibitor. In some aspects, the ion channel inhibitor indirectly interacts with an ion channel present in or on a cell when the cell is contacted with the ion channel inhibitor. In some aspects, these phrases can be used interchangeably. The contacting can be in vitro or in vivo. For example, if the sample is a petri dish/plate comprising cancer cells then the contacting occurs in vitro. However, if the sample is a human comprising cancer cells then the contacting occurs in vivo. In some aspects, if the contacting occurs in vivo then “contacting the cancer cell with an ion channel inhibitor” can be used interchangeably with “administering an ion channel inhibitor to a subject.” In some aspects, contacting a cancer cell with a chloride ion channel inhibitor can be used interchangeably with “exposing a cancer cell to a chloride ion channel inhibitor.”

In some aspects, the ion channel inhibitor is a chloride ion channel inhibitor. In some aspects, the chloride ion channel inhibitor is a CLIC inhibitor. In some aspects, the CLIC inhibitor is a CLIC1 or CLIC4 inhibitor. In some aspects, a chloride ion channel inhibitor can be, but is not limited to, Niflumic acid, DPC, DIDS, anthracene derivatives, stilbenes, and heavy metal ions blockbenzimidazole, Clofubric acid, Benzofurans, propionic acid (CPP), SITS, NPPB, Chlorotoxin, Mibefradil, Calix[4]arene, Clomiphene, Cd2+, Gd3+, Glibenclaimide, Flufenamic acid, Inositol-tetrabisphosphate, Mefloquinand or Fluoxetine.

In some aspects, the CLIC inhibitor can be a synthetic small molecule, protein, peptide, antibody, aptamer or a nucleic acid. For example, in some aspects, the CLIC inhibitor is a small molecule, such as IAA94. In some aspects, the CLIC inhibitor is a siRNA. In some aspects, the siRNA inhibits the expression of a protein selected from the group consisting of CLIC1, CLIC4, CLIC2, CLIC3, CLIC5, and CLIC6. In some aspects, the siRNA can be one or more of GGAGAUCGAGCUCGCCUAU (SEQ ID NO:1), CAUCGGUACUUGAGCAAUG (SEQ ID NO:2), GGCAAAGGCCCUCAAAUAA (SEQ ID NO:3), GGACCGAGACAGUGCAGAA (SEQ ID NO:4), CCAAAAGACUCACCAAGUA (SEQ ID NO:5), CCUCAUAGCUUAAAGUAUA (SEQ ID NO:6), UGACUUAGCUAUAGCAGUA (SEQ ID NO:7), or GACAAAAGCUAGCUAGUAA (SEQ ID NO:8), or the complement thereof. In some aspects, the siRNA can be a sequence comprising 80%, 85%, 90%, 95%, 99% or any percent in-between, identity or complementarity to one or more GGAGAUCGAGCUCGCCUAU (SEQ ID NO:1), CAUCGGUACUUGAGCAAUG (SEQ ID NO:2), GGCAAAGGCCCUCAAAUAA (SEQ ID NO:3), GGACCGAGACAGUGCAGAA (SEQ ID NO:4), CCAAAAGACUCACCAAGUA (SEQ ID NO:5), CCUCAUAGCUUAAAGUAUA (SEQ ID NO:6), UGACUUAGCUAUAGCAGUA (SEQ ID NO:7), or GACAAAAGCUAGCUAGUAA (SEQ ID NO:8).

In some aspects, the cancer cell is contacted with the ion channel inhibitor prior to exposing the cell to the alternating electric fields. In some aspects, contacting the cancer cell with the ion channel inhibitor prior to exposing the cancer cell to the alternating electric fields can refer to the cancer cell being contacted with the ion channel inhibitor at least 1, 2, 3, 4, 5, 6, 7, 8, 9, 10, 11, 12, 13, 14, 15, 16, 17, 18, 19, 20, 21, 22, 23, or 24 hrs prior to or at least 1, 2, 3, 4, 5, 6, 7, 8, 9, or 10 days prior to exposing the cell to the alternating electric fields. In some aspects, the cancer cell is contacted with the ion channel inhibitor after exposing the cell to the alternating electric fields. In some aspects, contacting the cancer cell with the ion channel inhibitor after exposing the cancer cell to the alternating electric fields can refer to the cancer cell being contacted with the ion channel inhibitor at least 1, 2, 3, 4, 5, 6, 7, 8, 9, 10, 11, 12, 13, 14, 15, 16, 17, 18, 19, 20, 21, 22, 23, or 24 hrs after or at least 1, 2, 3, 4, 5, 6, 7, 8, 9, or 10 days after exposing the cancer cell to the alternating electric fields. In some aspects, contacting the cancer cell with the ion channel inhibitor simultaneously with exposing the cells to the alternating electric fields. In some aspects, exposing the cell to the alternating electric fields simultaneously can mean applying within 5, 10, 15, 20, 25, 30, 35, 40, 45, 50, 55, or 60 minutes before or after contacting the cancer cell with the ion channel inhibitor.

In some aspects, the cancer is glioblastoma. In some aspects, the cancer can be, but is not limited to, brain cancer, breast cancer, ovarian cancer, lung cancer, colon cancer, prostate cancer, sarcoma, colorectal cancer, liver cancer, skin cancer, or stomach cancer.

In some aspects, the frequency of the alternating electric field is between 50 and 1 MHz. In some aspects, the frequency of the alternating electric field is 100-500 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 1 V/cm RMS. In some aspects, the alternating electric field has a field strength of any of those described herein.

### F. Methods of Reducing Cancer Cell Proliferation

An important role for ion channels in tumor development and growth has been defined. Chloride channels regulate cell cycle and are over expressed in cancer. In some aspects, CLIC1 and CLIC4 are overexpressed following alternating electric fields application in cancer cell lines. In some aspects, specific inhibitors of CLIC1 and CLIC4 can be used in treating cancer and inhibiting cell growth. In some aspects, disclosed herein is the combination of alternating electric fields with CLIC1 and CLIC4 inhibitors which results in combined cytotoxic effect in cancer cell lines.

In some aspects, the disclosed methods comprise contacting a cell with an ion channel inhibitor or a composition comprising an ion channel inhibitor as described throughout.

Disclosed are methods of reducing cancer cell proliferation comprising exposing the cancer cell to alternating electric fields for a period of time, the alternating electric fields having a frequency and field strength, and contacting the cancer cell with an ion channel inhibitor wherein the alternating electric fields and ion channel inhibitor provide an anti-proliferative effect of the cancer cell. Specifically, disclosed are methods of reducing cancer cell proliferation comprising exposing the cancer cell to alternating electric fields for a period of time, the alternating electric fields having a frequency and field strength, and contacting the cancer cell with a chloride ion channel inhibitor wherein the alternating electric fields and chloride ion channel inhibitor provide an anti-proliferative effect of the cancer cell.

In some aspects, contacting a cancer cell with a chloride ion channel inhibitor can include adding a chloride ion channel inhibitor to a sample comprising one or more cancer cells. In some aspects, contacting a cancer cell with a chloride ion channel inhibitor can include adding one or more cancer cells to a sample comprising a chloride ion channel inhibitor. Thus, in some aspects, a cancer cell is contacting an ion channel inhibitor and in some aspects, the ion channel inhibitor is contacting a cancer cell.

In some aspects, the ion channel inhibitor directly interacts with an ion channel present in or on a cell when the cell is contacted with the ion channel inhibitor. In some aspects, the ion channel inhibitor indirectly interacts with an ion channel present in or on a cell when the cell is contacted with the ion channel inhibitor. In some aspects, these phrases can be used interchangeably. The contacting can be in vitro or in vivo. For example, if the sample is a petri dish/plate comprising cancer cells then the contacting occurs in vitro. However, if the sample is a human comprising cancer cells then the contacting occurs in vivo. In some aspects, if the contacting occurs in vivo then “contacting the cancer cell with an ion channel inhibitor” can be used interchangeably with “administering an ion channel inhibitor to a subject.” In some aspects, contacting a cancer cell with a chloride ion channel inhibitor can be used interchangeably with “exposing a cancer cell to a chloride ion channel inhibitor.”

In some aspects, the ion channel inhibitor is a chloride ion channel inhibitor. In some aspects, the chloride ion channel inhibitor is a CLIC inhibitor. In some aspects, the CLIC inhibitor is a CLIC1 or CLIC4 inhibitor. In some aspects, a chloride ion channel inhibitor can be, but is not limited to, Niflumic acid, DPC, DIDS, anthracene derivatives, stilbenes, and heavy metal ions blockbenzimidazole, Clofubric acid, Benzofurans, propionic acid (CPP), SITS, NPPB, Chlorotoxin, Mibefradil, Calix[4]arene, Clomiphene, Cd2+, Gd3+, Glibenclaimide, Flufenamic acid, Inositol-tetrabisphosphate, Mefloquinand or Fluoxetine.

In some aspects, the CLIC inhibitor can be a synthetic small molecule, protein, peptide, antibody, aptamer or a nucleic acid. For example, in some aspects, the CLIC inhibitor is a small molecule, such as IAA94. In some aspects, the CLIC inhibitor is a siRNA. In some aspects, the siRNA inhibits the expression of a protein selected from the group consisting of CLIC1, CLIC4, CLIC2, CLIC3, CLIC5, and CLIC6. In some aspects, the siRNA can be one or more of GGAGAUCGAGCUCGCCUAU (SEQ ID NO:1), CAUCGGUACUUGAGCAAUG (SEQ ID NO:2), GGCAAAGGCCCUCAAAUAA (SEQ ID NO:3), GGACCGAGACAGUGCAGAA (SEQ ID NO:4), CCAAAAGACUCACCAAGUA (SEQ ID NO:5), CCUCAUAGCUUAAAGUAUA (SEQ ID NO:6), UGACUUAGCUAUAGCAGUA (SEQ ID NO:7), or GACAAAAGCUAGCUAGUAA (SEQ ID NO:8), or the complement thereof. In some aspects, the siRNA can be a sequence comprising 80%, 85%, 90%, 95%, 99% or any percent in-between, identity or complementarity to one or more GGAGAUCGAGCUCGCCUAU (SEQ ID NO:1), CAUCGGUACUUGAGCAAUG (SEQ ID NO:2), GGCAAAGGCCCUCAAAUAA (SEQ ID NO:3), GGACCGAGACAGUGCAGAA (SEQ ID NO:4), CCAAAAGACUCACCAAGUA (SEQ ID NO:5), CCUCAUAGCUUAAAGUAUA (SEQ ID NO:6), UGACUUAGCUAUAGCAGUA (SEQ ID NO:7), or GACAAAAGCUAGCUAGUAA (SEQ ID NO:8).

In some aspects, the cancer cell is contacted with the ion channel inhibitor prior to exposing the cell to the alternating electric fields. In some aspects, contacting the cancer cell with the ion channel inhibitor prior to exposing the cancer cell to the alternating electric fields can refer to the cancer cell being contacted with the ion channel inhibitor at least 1, 2, 3, 4, 5, 6, 7, 8, 9, 10, 11, 12, 13, 14, 15, 16, 17, 18, 19, 20, 21, 22, 23, or 24 hrs prior to or at least 1, 2, 3, 4, 5, 6, 7, 8, 9, or 10 days prior to exposing the cell to the alternating electric fields. In some aspects, the cancer cell is contacted with the ion channel inhibitor after exposing the cell to the alternating electric fields. In some aspects, contacting the cancer cell with the ion channel inhibitor after exposing the cancer cell to the alternating electric fields can refer to the cancer cell being contacted with the ion channel inhibitor at least 1, 2, 3, 4, 5, 6, 7, 8, 9, 10, 11, 12, 13, 14, 15, 16, 17, 18, 19, 20, 21, 22, 23, or 24 hrs after or at least 1, 2, 3, 4, 5, 6, 7, 8, 9, or 10 days after exposing the cancer cell to the alternating electric fields. In some aspects, contacting the cancer cell with the ion channel inhibitor simultaneously with exposing the cells to the alternating electric fields. In some aspects, exposing the cell to the alternating electric fields simultaneously can mean applying within 5, 10, 15, 20, 25, 30, 35, 40, 45, 50, 55, or 60 minutes before or after contacting the cancer cell with the ion channel inhibitor.

In some aspects, the cancer is glioblastoma. In some aspects, the cancer can be, but is not limited to, brain cancer, breast cancer, ovarian cancer, lung cancer, colon cancer, prostate cancer, sarcoma, colorectal cancer, liver cancer, skin cancer, or stomach cancer.

### G. Methods of Increasing Survival Rate

In some aspects, the combination of alternating electric fields and an ion channel inhibitor can increase the survival rate of a subject having cancer. In some aspects, the increased survival rate can be due to the killing of cancer cells or the reduction of proliferation of cancer cells, thus preventing the spread of the cancer.

The disclosed methods comprise administering an ion channel inhibitor or a composition comprising an ion channel inhibitor as described throughout. In some aspects, the ion channel inhibitor is a chloride ion channel inhibitor.

Disclosed are methods of increasing survival rate of a subject having cancer comprising applying alternating electric fields to a target site of the subject for a period of time, the alternating electric fields having a frequency and field strength, wherein the target site comprises one or more cancer cells, and administering an ion channel inhibitor to the subject, wherein the subject has an increased survival rate. Specifically, disclosed are methods of increasing survival rate of a subject having cancer comprising applying alternating electric fields to a target site of the subject for a period of time, the alternating electric fields having a frequency and field strength, wherein the target site comprises one or more cancer cells, and administering a chloride ion channel inhibitor to the subject, wherein the subject has an increased survival rate.

In some aspects, the ion channel inhibitor is a chloride ion channel inhibitor. In some aspects, the chloride ion channel inhibitor is an intracellular chloride ion channel (CLIC) inhibitor. In some aspects, a chloride ion channel inhibitor can be, but is not limited to, Niflumic acid, DPC, DIDS, anthracene derivatives, stilbenes, and heavy metal ions blockbenzimidazole, Clofubric acid, Benzofurans, propionic acid (CPP), SITS, NPPB, Chlorotoxin, Mibefradil, Calix[4]arene, Clomiphene, Cd2+, Gd3+, Glibenclaimide, Flufenamic acid, Inositol-tetrabisphosphate, Mefloquinand or Fluoxetine. In some aspects, the CLIC inhibitor is a CLIC1 or CLIC4 inhibitor. CLIC1 and CLIC 4 have been noted as potential biomarkers in ovarian and colorectal cancer and are over expressed in many tumours, linking to poor overall survival. CLIC1 and CLIC are also linked to migratory capacity of glioblastoma cells. Therefore, inhibiting CLIC1 and CLIC4 in combination with treating with alternating electric fields can be an effective therapeutic.

In some aspects, the CLIC inhibitor can be a synthetic small molecule, protein, peptide, antibody, aptamer or a nucleic acid. For example, in some aspects, the CLIC inhibitor can be a small molecule, such as IAA94. In some aspects, the CLIC inhibitor is a siRNA. wherein the siRNA inhibits the expression of a protein selected from the group consisting of CLIC1, CLIC4, CLIC2, CLIC3, CLIC5, and CLIC6. In some aspects, the siRNA can be one or more of GGAGAUCGAGCUCGCCUAU (SEQ ID NO:1), CAUCGGUACUUGAGCAAUG (SEQ ID NO:2), GGCAAAGGCCCUCAAAUAA (SEQ ID NO:3), GGACCGAGACAGUGCAGAA (SEQ ID NO:4), CCAAAAGACUCACCAAGUA (SEQ ID NO:5), CCUCAUAGCUUAAAGUAUA (SEQ ID NO:6), UGACUUAGCUAUAGCAGUA (SEQ ID NO:7), or GACAAAAGCUAGCUAGUAA (SEQ ID NO:8), or the complement thereof. In some aspects, the siRNA can be a sequence comprising 80%, 85%, 90%, 95%, 99% or any percent in-between, identity or complementarity to one or more GGAGAUCGAGCUCGCCUAU (SEQ ID NO:1), CAUCGGUACUUGAGCAAUG (SEQ ID NO:2), GGCAAAGGCCCUCAAAUAA (SEQ ID NO:3), GGACCGAGACAGUGCAGAA (SEQ ID NO:4), CCAAAAGACUCACCAAGUA (SEQ ID NO:5), CCUCAUAGCUUAAAGUAUA (SEQ ID NO:6), UGACUUAGCUAUAGCAGUA (SEQ ID NO:7), or GACAAAAGCUAGCUAGUAA (SEQ ID NO:8).

In some aspects, the ion channel inhibitor can be administered prior to applying the alternating electric fields. In some aspects, administering the ion channel prior to applying the alternating electric fields can refer to administering the ion channel inhibitor at least 1, 2, 3, 4, 5, 6, 7, 8, 9, 10, 11, 12, 13, 14, 15, 16, 17, 18, 19, 20, 21, 22, 23, or 24 hrs prior to or at least 1, 2, 3, 4, 5, 6, 7, 8, 9, or 10 days prior to applying the alternating electric fields. In some aspects, the ion channel inhibitor can be administered after applying the alternating electric fields. In some aspects, administering the ion channel after applying the alternating electric fields can refer to administering the ion channel inhibitor at least 1, 2, 3, 4, 5, 6, 7, 8, 9, 10, 11, 12, 13, 14, 15, 16, 17, 18, 19, 20, 21, 22, 23, or 24 hrs after or at least 1, 2, 3, 4, 5, 6, 7, 8, 9, or 10 days after applying the alternating electric fields. In some aspects, the ion channel inhibitor is administered simultaneously with applying the alternating electric fields. In some aspects, applying the alternating electric fields simultaneously can mean applying within 5, 10, 15, 20, 25, 30, 35, 40, 45, 50, 55, or 60 minutes before or after administering an ion channel inhibitor.

In some aspects, the alternating electric fields and ion channel inhibitor increase overall survival of the subject. In some aspects, the alternating electric fields and ion channel inhibitor reduce proliferation of the one or more cancer cells. In some aspects, the alternating electric fields and ion channel inhibitor reduce migration of the one or more cancer cells. The decrease in proliferation and/or migration of cancer cells can result in a less invasive cancer.

In some aspects, the cancer is glioblastoma. In some aspects, the cancer can be, but is not limited to, brain cancer, breast cancer, ovarian cancer, lung cancer, colon cancer, prostate cancer, sarcoma, colorectal cancer, liver cancer, skin cancer, or stomach cancer.

In some aspects, the frequency of the alternating electric field is between 50 and 1 MHz. In some aspects, the frequency of the alternating electric field is 100-500 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 1 V/cm RMS. In some aspects, the alternating electric field has a field strength of any of those described herein.

### H. Combination Therapy with Alternating Electric Fields, Ion Channel Inhibitor, and Second Therapeutic

In some aspects, any of the disclosed methods can further comprise a step of administering a second therapeutic.

For example, disclosed are methods of treating a subject having cancer comprising applying alternating electric fields to a target site of the subject for a period of time, the alternating electric fields having a frequency and field strength, wherein the target site comprises one or more cancer cells, and administering a chloride ion channel inhibitor to the subject, and further comprising administering a second cancer therapeutic to the subject.

Also disclosed are methods of increasing survival rate of a subject having cancer comprising applying alternating electric fields to a target site of the subject for a period of time, the alternating electric fields having a frequency and field strength, wherein the target site comprises one or more cancer cells, and administering a chloride ion channel inhibitor to the subject, and further comprising administering a second cancer therapeutic to the subject, wherein the subject has an increased survival rate.

Disclosed are methods of increasing survival rate of a subject having cancer comprising applying alternating electric fields to a target site of the subject for a period of time, the alternating electric fields having a frequency and field strength, wherein the target site comprises one or more cancer cells, and administering an ion channel inhibitor to the subject and further comprising administering a second cancer therapeutic to the subject, wherein the subject has an increased survival rate.

Disclosed are methods of killing a cancer cell comprising exposing the cancer cell to alternating electric fields for a period of time, the alternating electric fields having a frequency and field strength, and contacting the cancer cell with an ion channel inhibitor, and further comprising contacting the cancer cell with a second cancer therapeutic to the subject, wherein the subject has an increased survival rate and wherein cancer cells are killed.

Disclosed are methods of reducing cancer cell proliferation comprising exposing the cancer cell to alternating electric fields for a period of time, the alternating electric fields having a frequency and field strength, and contacting the cancer cell with an ion channel and a second cancer therapeutic, wherein the alternating electric fields, ion channel inhibitor and second cancer therapeutic provide an anti-proliferative effect of the cancer cell.

In some aspects, a second cancer therapeutic can be a chemotherapeutic agent. In some aspects, the chemotherapeutic agent can be dependent on the type of cancer the subject has. For example, if the subject has glioblastoma, the chemotherapeutic agent can be temozolomide. In some aspects, a second cancer therapeutic can be radiation.

### I. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more chloride channel inhibitors and one or more materials for delivering alternating electric fields, such as the Optune system. For example disclosed are kits comprising one or more CLIC1 or CLIC4 inhibitors and one or more materials for delivering alternating electric fields, such as the Optune system. In some aspects, the kit can further comprise a second therapeutic.

### EXAMPLES

### A. Example 1

Glioblastoma multiforme (GBM) is a lethal brain cancer with an average survival of 14 months, forming the leading cause of CNS cancer-related death in children. There are currently no curative treatments.

Tumour treating fields (TTFields) are a non-invasive antimitotic therapy approved in GBM patients>22 years. Previous studies have

identified the clinical benefit of TTFields; however, the mechanistic action was unclear. TTFields can deliver

200 kHz alternating electric fields to the tumour.

Ion channels are essential in cell cycle control, invasion and migration of cancer cells and therefore present as valuable therapeutic targets.

CLIC1 and CLIC4 are potential biomarkers in ovarian and colorectal cancer and are over-expressed in many tumours, linking to poor overall survival. They are also linked to migratory capacity of glioblastoma cells. CLIC1 and CLIC4 are essential in cell cycle progression, proliferation and invasion in many cancers. CLIC1 and CLIC4 have a dual role as both soluble membrane proteins and globular proteins, therefore two actions to target.

Chloride intracellular channels (CLICs) can be a therapeutic target due to their intrinsic individualities that allow focus on cancer cells and not healthy cells. CLIC1 and CLIC4, in particular, not only are overexpressed in specific tumor types or their corresponding stroma but also change localization and function from hydrophilic cytosolic to integral transmembrane proteins as active ionic channels or signal transducers during cell cycle progression in certain cases. These changes in intracellular localization, tissue compartments, and channel function, uniquely associated with malignant transformation, can offer a unique target for cancer therapy, likely able to spare normal cells.

This study characterizes CLIC1 and CLIC4 channels in paediatric glioblastoma (pGBM). Genetic and pharmacological inhibition of CLIC1 and CLIC4 ion channels was used to reduce the capacity of pGBM to proliferate, migrate and invade. This study also analyzes the pathway(s) involved in electrotherapy. The study shows the efficacy of TTFields and deep brain stimulation could be exacerbated via ion channel inhibition.

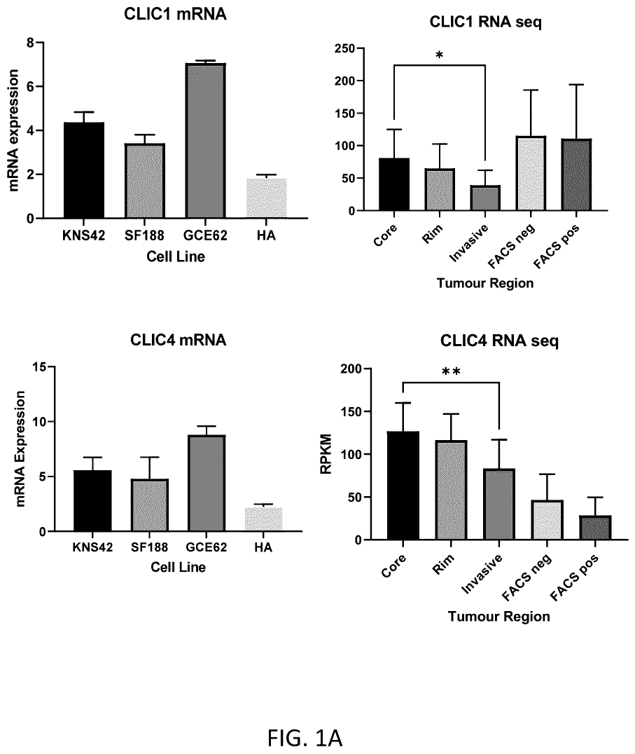

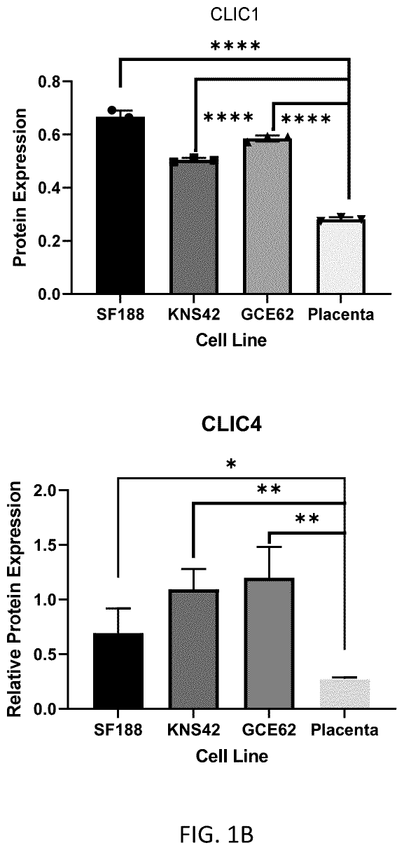

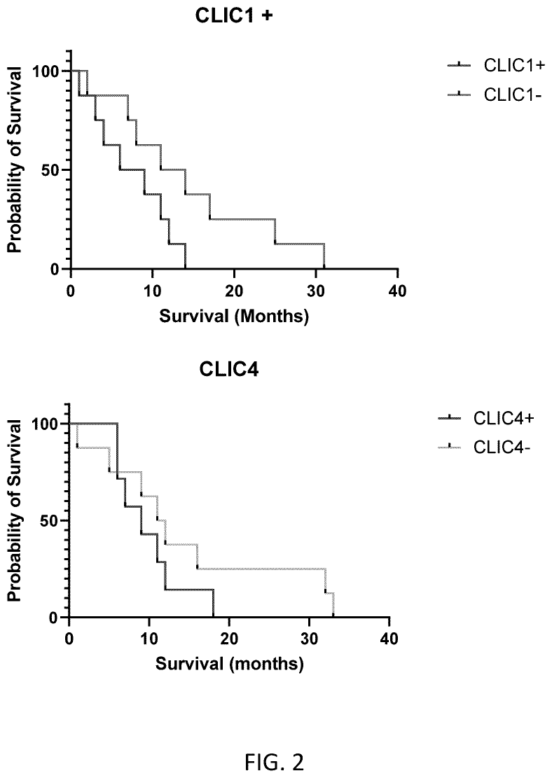

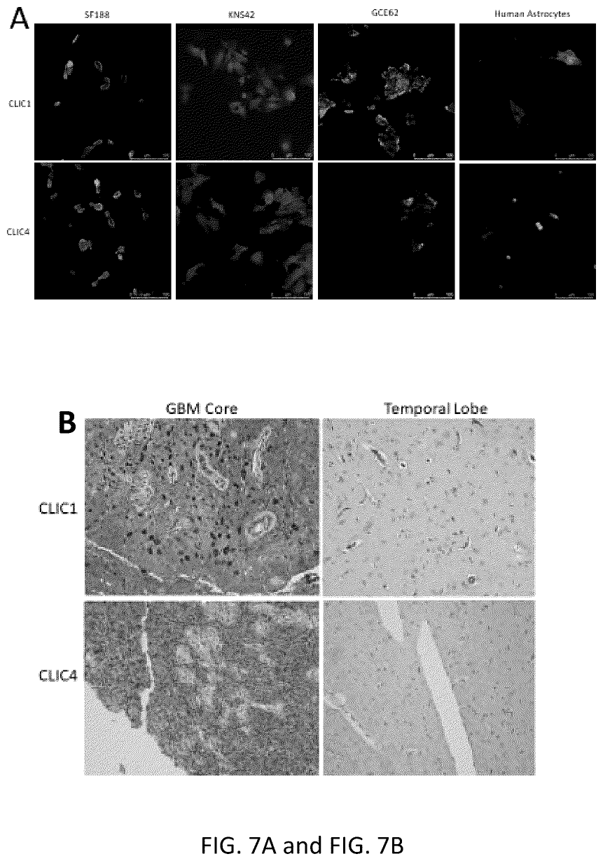

CLIC1 and CLIC4 are prognostic markers for overall survival in paediatric and adult GBM cohorts, with over expression conferring to poor overall survival (FIGS. 1, 2, and 7). FIG. 1 shows high expression of CLIC1 and CLIC4, with particular accumulation in the core regions of the tumor. FIG. 2 is an example of a Kaplan Meier analysis of in house pediatric and adult (data not shown) high grade glioma patient tumor samples that was carried out on tissue microarrays stained via IHC for either CLIC1 or CLIC4. Overexpression of CLIC1 and CLIC4 in the patient group confers poor outcome with low expression being favorable for overall survival. FIG. 7 shows that CLIC1 and CLIC4 are significantly upregulated in adult and pGBM patient tissue samples.

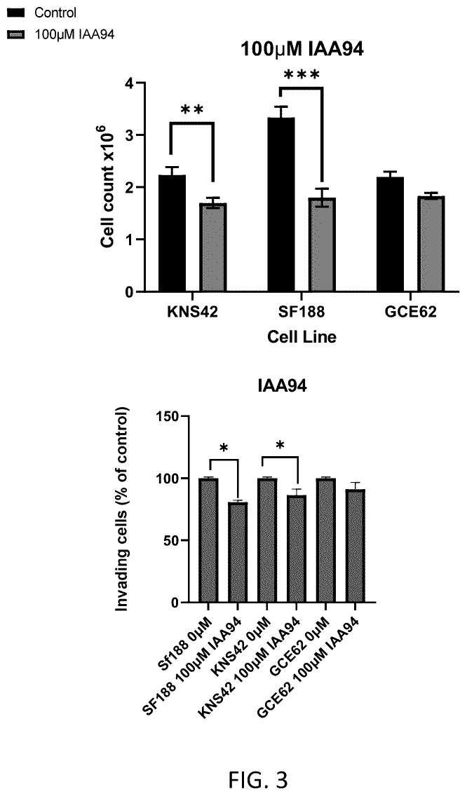

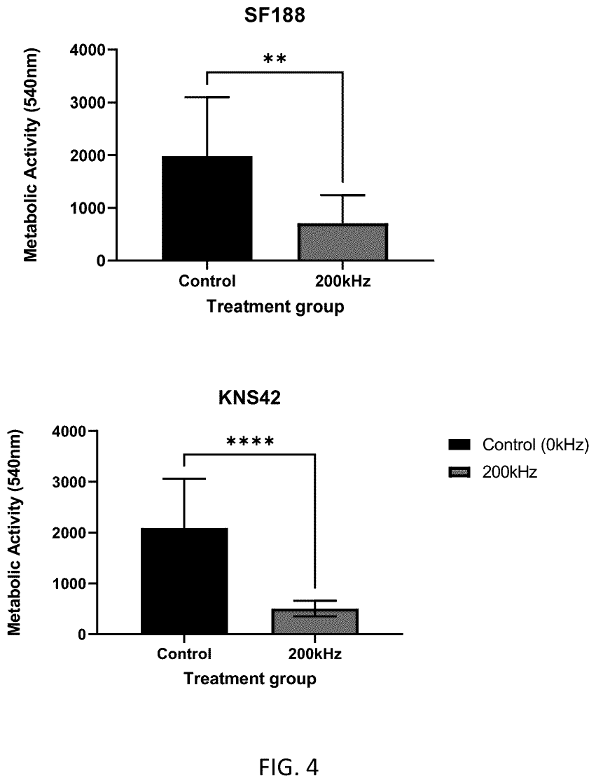

The inhibition of CLIC1 and CLIC4 by IAA94 reduced the capacity of pGBM to proliferate, invade and migrate (FIG. 3). Pharmacological inhibition via treatment with 100 uM IAA94 reduces overall cell count and reduces invasive capacity in pGBM. Similar efficacy was observed when using TTFields as a treatment for pGBM (FIG. 4).

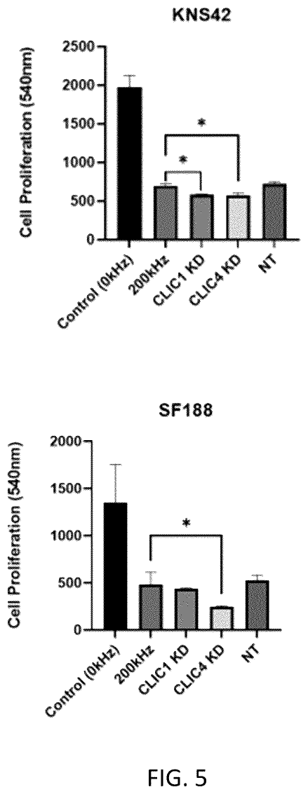

FIG. 5 shows a decrease in cell proliferation in KNS42 and SF188 cells treated with TTFields. The combination of TTFields plus the knock down of CLIC1 or CLIC4 significantly decreased cell proliferation compared to TTFields alone.

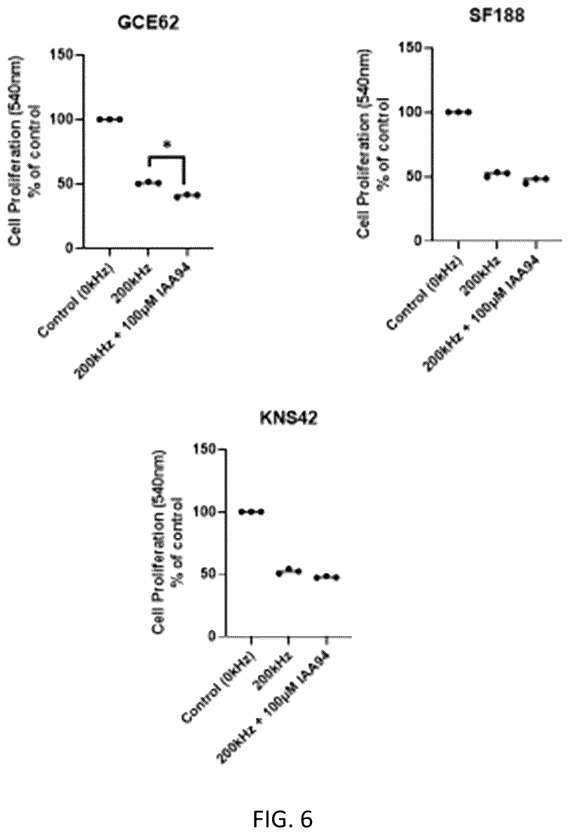

FIG. 6 shows treatment of three cell lines (GCE62, SF188 and KNS42) with TTFields and IAA94. The addition of TTFields alone greatly reduces the cell proliferation and the combination of TTFields and IAA94 significantly reduced cell proliferation compared to TTFields alone.

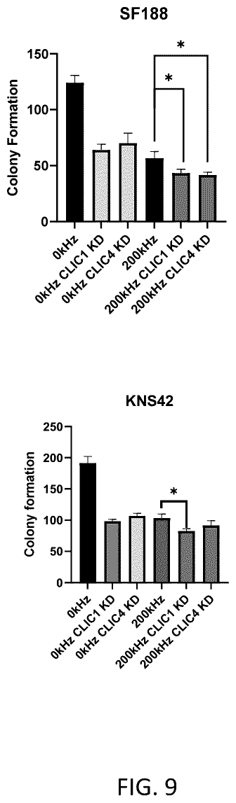

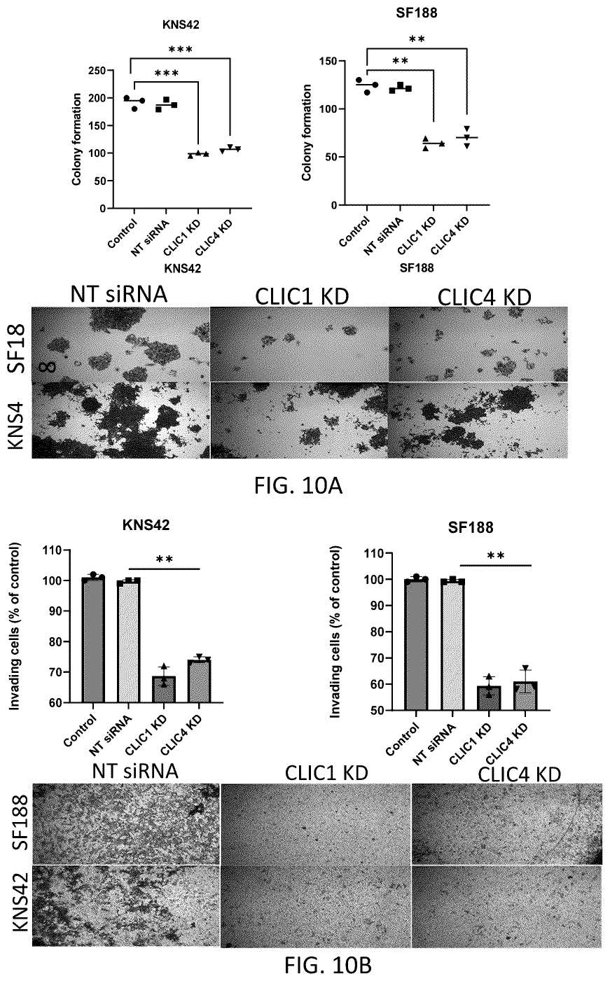

FIGS. 5 and 6 thus evidence that both genetic inhibition and pharmacological inhibition of CLIC1 and CLIC4 reduces cell proliferation in cancer cell lines. Like FIG. 5, FIG. 9 shows CLIC1 and CLIC4 deficiency increases sensitivity to TTFields.

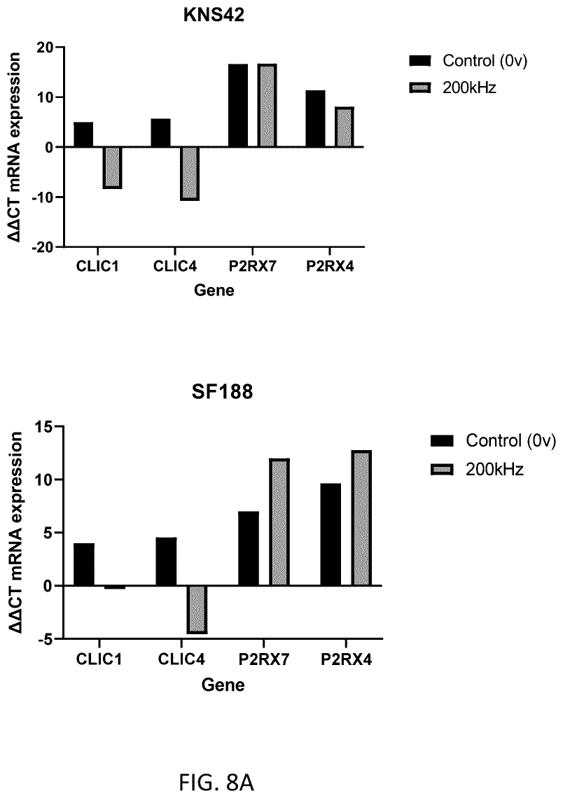

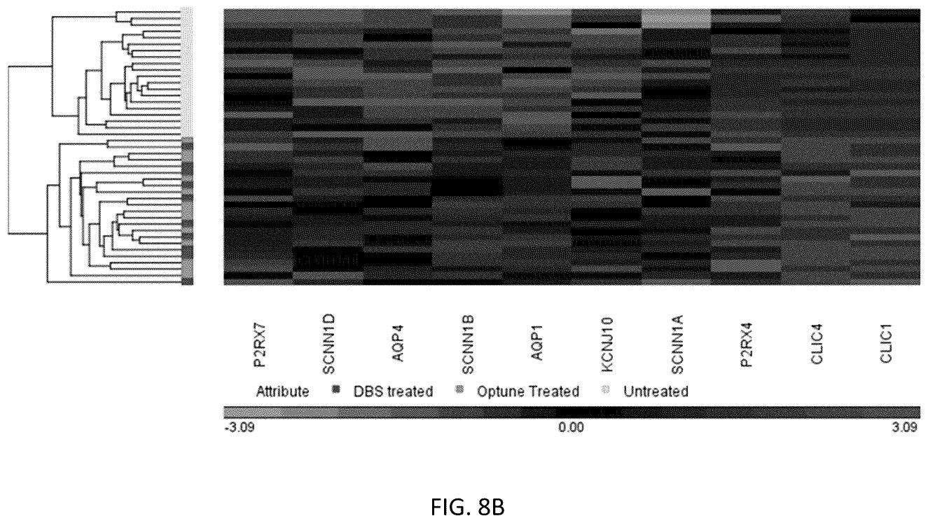

FIG. 8 shows treatment with electrotherapy causes CLIC1 and CLIC4 deficiency. CLIC1 and CLIC4 are significantly down regulated in treatment groups vs controls.

These data provide rationale that genetic, electrical, and pharmacological manipulation of ion channels reduces the capacity of childhood brain tumors to proliferate and invade. Low expression of CLIC1 and CLIC4 confers a survival benefit and can be used as a prognostic marker in pGBM. CLIC1 and CLIC4 deficiency increases sensitivity to TTFields and can be a target for combination therapy to enhance the treatment efficacy of TTFields.

### B. Example 2

The following data shows synergic antitumoral activity of TTFields stimulation and CLIC1 inhibition in human glioblastoma primary culture.

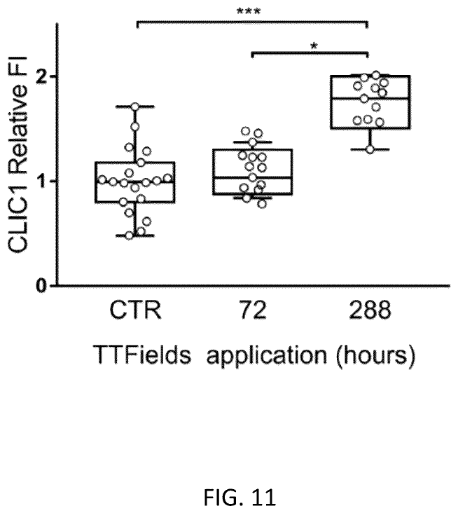

FIG. 11 shows an increase in CLIC1 mRNA expression after 288 hours of TTFields application compared to a control or compared to 72 hours of TTFields.

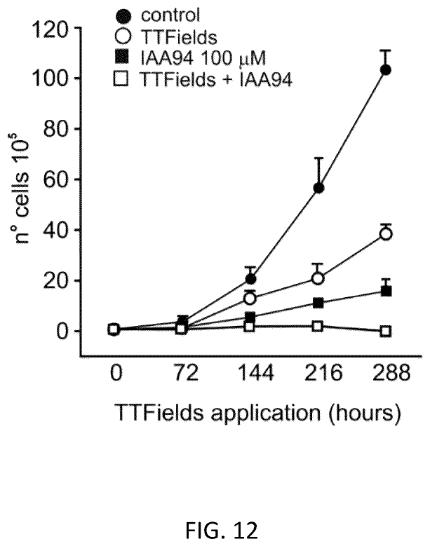

FIG. 12 shows that the combination of TTFields plus the CLIC1 inhibitor, IAA94, inhibits cell proliferation and/or kills more cells that TTFields alone or IAA94 alone. Thus, the combination therapy is a very effective treatment.

Those skilled in the art will recognize, or be able to ascertain using no more than routine experimentation, many equivalents to the specific embodiments of the method and compositions described herein. Such equivalents are intended to be encompassed by the following claims.

1. A method of treating a subject having cancer comprising:
a) applying alternating electric fields to a target site of the subject for a period of time, the alternating electric fields having a frequency and field strength,
wherein the target site comprises one or more cancer cells, and
b) administering a chloride ion channel inhibitor to the subject.

2. The method of claim 1, wherein the chloride ion channel inhibitor is a chloride intracellular channel (CLIC) inhibitor.

3. The method of claim 2, wherein the CLIC inhibitor is a CLIC1 or CLIC4 inhibitor.

4. (canceled)

5. The method of claim 2, wherein the CLIC inhibitor is IAA94

6. The method of claim 2, wherein the CLIC inhibitor is a siRNA.

7. The method of claim 6, wherein the siRNA inhibits the expression of a protein selected from the group consisting of CLIC1, CLIC4, CLIC2, CLIC3, CLIC5, and CLIC6.

8. (canceled)

9. The method of claim 1, wherein the alternating electric fields and chloride ion channel inhibitor reduces proliferation of the one or more cancer cells.

10. The method of claim 1, wherein the cancer is glioblastoma.

11. The method of claim 1, wherein the chloride ion channel inhibitor is administered prior to applying the alternating electric fields.

12. The method of claim 1, wherein the chloride ion channel inhibitor is administered after applying the alternating electric fields or simultaneously with applying the alternating electric fields.

13. (canceled)

14. A method of killing a cancer cell or reducing cancer cell proliferation comprising:
a) exposing the cancer cell to alternating electric fields for a period of time, the alternating electric fields having a frequency and field strength, and
b) contacting the cancer cell with a chloride ion channel inhibitor.

15. The method of claim 14, wherein the chloride ion channel inhibitor is a chloride intracellular channel (CLIC) inhibitor

16. The method of claim 15, wherein the CLIC inhibitor is a CLIC1 or CLIC4 inhibitor.

17. (canceled)

18. The method of claim 14, wherein the CLIC inhibitor is IAA94 or is siRNA.

19. (canceled)

20. The method of claim 18, wherein the siRNA inhibits the expression of a protein selected from the group consisting of CLIC1, CLIC4, CLIC2, CLIC3, CLIC5, and CLIC6.

21. (canceled)

22. (canceled)

23. (canceled)

24. The method of claim 14, wherein the cancer cell is in a subject.

25.-46. (canceled)

47. The method of claim 1, wherein the frequency of the alternating electric fields is between 50 kHz and 1 MHz.

48. The method of claim 47, wherein the frequency of the alternating electric fields is 200 kHz.

49. The method of claim 1, wherein the field strength is between 0.5V/cm and 4V/cm.

50. The method of claim 49, wherein the field strength is 1V/cm.
